(19) United States
(12) Patent Application Publication
(10) Pub. No.: US 2009/0103796 A1
(43) Pub. Date: Apr. 23, 2009

(54) METHOD OF DISCRIMINATING BETWEEN RIGHT AND LEFT BREAST IMAGES AND BREAST RADIOGRAPHING SYSTEM

(75) Inventors: Eiichi Akagi, Tokyo (JP); Mamoru Umeki, Tokyo (JP); Wataru Motoki, Tokyo (JP)

Correspondence Address: CANTOR COLBURN, LLP, 20 Church Street, 22nd Floor, Hartford, CT, 06103, US

(73) Assignee: KONICA MINOLTA MEDICAL & GRAPHIC, INC., Tokyo (JP)

(21) Appl. No.: 11/915,803

(22) PCT Filed: May 19, 2006
(86) PCT No.: PCT/JP2006/310000

§ 371 Date: Nov. 28, 2007

(30) Foreign Application Priority Data
Jun. 2, 2005 (JP) JP2005-162371
Jun. 2, 2005 (JP) JP2005-162377

(51) Int. Cl. G06K 9/00; A61B 6/00

(52) U.S. Cl. 382/132; 378/37

(57) ABSTRACT

The control apparatus sets the destination for sending data of the designated image reading apparatus and the breast image radiographing apparatus to be the control apparatus when receiving request for radiographing start in the mammography mode from the control apparatus. The breast image radiographing apparatus reads cassette ID of loaded cassette, and sends it to the control apparatus after correlating the cassette ID with UID indicating the radiographing time. The image reading apparatus reads cassette ID of loaded cassette and sends it along with the read image data to the control apparatus. The control apparatus correlates the received image data with the UID by using the cassette ID as a key, discriminates between MLO-L image and MLO-R image, and discriminates CC-L and CC-R images based on the discrimination result and the radiographing order indicated by the UID.

### FIELD OF THE INVENTION

The present invention relates to a method of discriminating between the right and left breast images in a medical radiographing system for radiographing the right and left breasts of one subject from a plurality of directions using a cassette.

### BACKGROUND OF THE INVENTION

An image obtained by using radiation is extensively used as a medical image for diagnosis. In recent years, digitization of the medical image has been achieved. For example, in the CR (Computed Radiography) apparatus, the radiation having passed through a subject is absorbed by a stimulable phosphor plate on which a stimulable phosphor layer is formed. After that, this stimulable phosphor plate is scanned by a laser beam, and the radiation energy stored by the phosphor layer is emitted as fluorescence, which is subjected to photoelectric conversion, whereby an image signal is obtained.

A radiographing system using this stimulable phosphor can be broadly classified into two categories as a special-purpose type incorporating a stimulable phosphor plate for both radiographing and image reading and a cassette type using a portable cassette incorporating a stimulable phosphor plate.

The cassette type radiographing system includes a radiographing apparatus for capturing the medical image of a subject using a cassette; an image reading apparatus for reading the image from the cassette; and a control apparatus for controlling this image reading apparatus to acquire the medical image having been read out. The control apparatus can communicate with the HIS (Hospital Information System) or RIS (Radiology Information System).

In such a radiographing system, the patient information such as information on the name and sex of the subject to be radiographed; the radiographing condition information such as information on the region and direction of radiographing; and the radiographing order information such as inspection information are issued prior to radiographing according to a doctor's request through the HIS or RIS. Such information is sent to the control apparatus. In the control apparatus, a list of radiographing order information is displayed at the time of radiographing.

The following describes the work flow of radiographing in this radiographing system:

1) The radiographing technician selects the radiographing order information out of the radiographing order information listed and displayed in the control apparatus.

2) To define the correspondence between the cassette to be used in radiographing and the radiographing order information, the radiographing technician follows the step of cassette registration. The cassette is provided with identification information (hereinafter referred to as “cassette ID”) to identify a particular cassette from other ones. In the cassette registration step, the cassette ID of the cassette to be corresponded to the selected radiographing order information is inputted into the control apparatus. In the control apparatus, the selected radiographing order information is displayed and the selected radiographing order information is correlated with the inputted cassette ID.

3) Based on the displayed radiographing order information, the radiographing technician checks the subject to be radiographed and radiographing conditions. After moving into a radiographing room with the registered cassette, the radiographing technician uses the radiographing apparatus to take a radiation image. In the radiographing apparatus, radiation is applied and the radiation image is recorded on the cassette.

4) The radiographing technician loads the cassette on the image reading apparatus so that reading operation starts. In the image reading apparatus, the medical image and cassette ID are read out of the cassette, and medical image and cassette ID having been read are correlated with each other to be sent to the control apparatus.

5) In the control apparatus, the radiographing order information is correlated with the medical image sent from the image reading apparatus, based on the cassette ID. In the final step, the radiographing order information is correlated with the medical image, and this information is sent to the HIS or RIS as the information accompanying the medical image.

The aforementioned radiographing system is disclosed in the Patent Document 1 wherein a plurality of control apparatuses and a plurality of image reading apparatuses are connected on the network. Even if the cassette having been registered with a control apparatus is read by any image reading apparatus, the medical image having been read is sent automatically back to the control apparatus having registered the cassette. In the radiographing system of the Patent Document 1, the radiographing technician is allowed to register a cassette using the nearest control apparatus, out of a plurality of control apparatuses. Even if the cassette has been read by a plurality of image reading apparatuses, he can go back to the control apparatus used for cassette registration, upon completion of the radiographing work in his charge, and can check the image. This arrangement enhances workflow efficiency.

Patent Document 1: Japanese Unexamined Patent Application Publication No. 2002-159476

### DISCLOSURE OF INVENTION

### Problems to be Solved by the Invention

Incidentally, in the diagnosis of a breast image, diagnosis is carried out by comparative radiographic interpretation of the right and left breast images and the images captured in different directions. Thus, at the time of mammography, it is a common practice to radiograph one subject a plurality of times under different radiographing conditions such as radiographing of the left breast in the vertical direction (CC (Cranio-Caudal)-L (left)), radiographing of the left breast in the oblique direction (MLO (Medio-Lateral Oblique)-L), radiographing of the right breast in the vertical direction (CC-R (right)) and radiographing of the right breast in the oblique direction (MLO-R). Accordingly, for mammography, the radiographing technician is required to register the cassette plural times for one subject in the aforementioned item (2).

In the aforementioned item (3), the radiographing technician is required to remember the radiographing conditions of radiographing order information correlated with each of the plural cassettes on the control apparatus and to pay attention not to confuse the correspondence between the radiographing conditions and the cassette. However, when many cassettes are brought into the radiographing room, they may be dropped, for example and this may increase the possibility of the cassettes being confused. Thus, the radiographing technician may perform radiographing work using the cassettes different from the ones registered to be radiographed under a certain radiographing condition so that the right and left breast identification error may result in the breast image captured.

An object of the present invention is to prevent the right and left breast of a subject from being confused in the breast images captured by a breast image radiographing system and to minimize the work load of the radiographing technician.

### Means for Solving the Problems

To solve the aforementioned problems, the method of discriminating between the right and left breast images is a discriminating method of plural breast images obtained by radiographing each of the right and left breasts of a subject described in Claim 1 including:

acquiring the information about the time of radiographing each of a plurality of the aforementioned breast images stored after correlated with one subject in advance;

determining the sequential order of radiographing each of a plurality of the aforementioned breast images, based on the acquired information about the time of radiographing;

discriminating between the right and left breasts of the subject in the pectoral muscle image captured while containing the pectoral muscle, out of a plurality of the aforementioned breast images; and

discriminating between the right and left of the breasts in each of a plurality of other breast images, based on the aforementioned radiographing sequential order, and the right and left discrimination of the breasts on the pectoral muscle image having been discriminated.

The method of discriminating between the right and left breast images described in Claim 2 is characterized in that, when radiographing the right and left breasts of one subject in the sequential order of alternately radiographing the right and left sides from a plurality of different radiographing directions, including a direction of the oblique direction for radiographing the images including the pectoral muscle, by using:

a breast radiographing system including:

a radiographing apparatus for radiographing a breast by applying radiation and recording the breast image on a cassette;

a control apparatus for processing the captured breast image; and

an image reading apparatus for reading the breast image recorded on the aforementioned cassette and sending the read breast image to the aforementioned control apparatus;

wherein they are connected in such a way as to permit data transmission and reception;

wherein, in the aforementioned radiographing apparatus, the identification information of the cassette used for each radiographing is read out; the read cassette identification information is correlated with the time information of the time of radiographing, and the resulting correlated information is sent to the aforementioned control apparatus;

in the aforementioned image reading apparatus, when the used cassette has been loaded, the identification information of the loaded cassette as well as the breast image recorded on the cassette is read and the read cassette identification information and the breast image are sent correlated with each other to the control apparatus; and

in the aforementioned control apparatus, upon receipt of a plurality of captured breast images of the subject and a plurality of pieces of time information, each of the received breast images is correlated with each of the pieces of time information based on the cassette identification information; the sequential order of radiographing a plurality of the aforementioned breast images is determined, based on plural pieces of the received time information of the breast images; discrimination is made between the right and left breasts of the subject in the pectoral muscle image captured while containing a pectoral muscle, out of a plurality of the aforementioned breast images; and discrimination is made between the right and left of the breasts of the subject in the breast images captured from other directions, based on the sequential order of radiographing a plurality of breast images and the right and left discrimination of the breasts on the pectoral muscle image having been discriminated.

The method of discriminating between the right and left breast images described in Claim 3 is characterized in that, in the invention described in Claim 1 or 2, by identifying the subject of a plurality of the aforementioned breast images, the right and left breasts of the subject are discriminated in at least one breast image captured while containing the pectoral muscle out of a plurality of the aforementioned breast images.

The invention described in Claim 4 is characterized in that, in the invention described in any one of the Claims 1, 2 and 3, right and left breast discrimination is made on the assumption that the side of breast among right and left of the breasts in the breast image having the radiographing sequential order following or followed by that of the breast image containing a pectoral muscle is different from that in the breast images containing a pectoral muscle.

The invention described in Claim 5 is characterized in that, in the invention described in Claim 2, a plurality of control apparatuses for processing the breast images captured by the aforementioned radiographing apparatus are connected, and a management apparatus controls the connection between the plurality of control apparatuses and the image reading apparatus.

The invention described in Claim 6 is characterized in that, in the invention described in Claim 5, the aforementioned management apparatus ensures that the information is sent to a predetermined control apparatus out of a plurality of control apparatuses.

The invention described in Claim 7 is characterized in that, in the invention described in Claim 5, the aforementioned management apparatus ensures that the information is sent to the control apparatus wherein the identification information of a plurality of the cassettes to be used for radiographing of the aforementioned subject from a plurality of radiographing directions has been registered.

The invention described in Claim 8 is characterized in that, in the invention described in any one of the Claims 5 through 7, a plurality of the aforementioned image reading apparatuses are connected to the aforementioned breast radiographing system.

The breast radiographing system described in Claim 9 is characterized by including:

a radiographing apparatus for radiographing a breast by applying radiation and recording the breast image on a cassette;

a control apparatus for processing the captured breast image; and

an image reading apparatus for reading the breast image recorded on the aforementioned cassette and sending the read breast image to the aforementioned control apparatus;

wherein they are connected in such a way as to permit data transmission and reception,

wherein when radiographing each of the right and left breasts of one subject in the sequential order of alternately radiographing the right and left sides from a plurality of different radiographing directions, including a direction of the oblique direction for radiographing the images containing a pectoral muscle,

wherein, in the aforementioned radiographing apparatus, the cassette identification information used for each radiographing is read out; the read cassette identification information is correlated with the time information of the time of radiographing, and the resulting correlated information is sent to the aforementioned control apparatus;

in the aforementioned image reading apparatus, when the used cassette has been loaded, the identification information of the loaded cassette as well as the breast image recorded on the cassette is read, and the read cassette identification information and the breast image are sent correlated with each other to the control apparatus, and the aforementioned control apparatus includes:

a receiving device for receiving a plurality of captured breast images of the subject and a plurality of pieces of time information;

a radiographing order determining device for correlating each of the received breast images with each of time information, based on the cassette identification information, and determining the sequential order of radiographing a plurality of the aforementioned breast images having been received; and

a right and left discrimination device for discriminating between the right and left breasts of the subject in the pectoral muscle image captured while containing a pectoral muscle, out of a plurality of the breast images, and for discriminating between the right and left of the breasts of the subject in the breast images captured from other directions, based on the sequential order of radiographing a plurality of breast images and the right and left discrimination of the breasts having been discriminated in the pectoral muscle image captured while containing the pectoral muscle.

The invention described in Claim 10 is characterized in that, in the invention described in Claim 9, the aforementioned right and left discrimination device includes a subject identification device for identifying the subject of a plurality of the aforementioned breast images, and discriminates between the right and left breasts of the subject in the breast images captured while including a pectoral muscle, out of a plurality of breast images.

The invention described in Claim 11 is characterized in that, in the invention described in Claim 9 or 10, the right and left discrimination device performs the step of right and left discrimination on the assumption that the side of breast among the right and left of the breasts of the subject in the breast image which has been captured from a different direction and which has the radiographing sequential order following or followed by that of the breast image containing a pectoral muscle is different from that in the breast images containing a pectoral muscle.

The invention described in Claim 12 is characterized in that, in the invention described in Claim 9, a plurality of control apparatuses are connected to apply processing to the breast image having been captured by the radiographing apparatus, and a management apparatus controls the connection between a plurality of the aforementioned control apparatuses and the aforementioned image reading apparatus.

The invention described in Claim 13 is characterized in that, in the invention described in Claim 12, the aforementioned management apparatus ensures that information is sent to a predetermined control apparatus out of a plurality of the aforementioned control apparatuses.

The invention described in Claim 14 is characterized in that, in the invention described in Claim 12, the aforementioned management apparatus ensures that information is sent to the control apparatus that has registered the identification information for a plurality of cassettes to be used for radiographing of the aforementioned subject from a plurality of radiographing directions.

The invention described in Claim 15 is characterized in that, in the invention described in any one of the Claims 12 through 14, a plurality of the aforementioned image reading apparatuses are connected to the breast radiographing system.

### EFFECTS OF THE INVENTION

According to the invention described in Claim 1, there are the steps of acquiring the information on the time of radiographing for each of a plurality of the aforementioned breast images stored while correlated with one subject in advance; discriminating the sequential order of radiographing each of a plurality of the aforementioned breast images, based on the acquired information on the time of radiographing; discriminating between the right and left breasts of the subject in the pectoral muscle image captured while containing the pectoral muscle, out of a plurality of the aforementioned breast images; and discriminating between the right and left of the breasts in each of a plurality of other breast images, based on the aforementioned radiographing sequential order, and the right and left discrimination of the breasts on the pectoral muscle image having been determined. This arrangement prevents the right and left breast of the subject from being confused in a captured breast image.

According to the invention described in Claims 2 and 9, control apparatuses are used to discriminating automatically between the right and left breasts of a subject in a plurality of breast images having been captured for one subject, thereby preventing the right and left breast of the subject from being confused in a radiographed breast image. Further, the radiographing technician is relieved of a burden of remembering the radiographing condition corresponding to each cassette to be used for radiographing, when radiographing a plurality of breast images of one subject, as in the conventional method. This signifies a substantial reduction of the work load of the radiographing technician.

According to the invention described in Claims 3 and 10, control apparatuses are used to automatically discriminate between the right and left breasts of the subject in the pectoral muscle image captured while containing the pectoral muscle, out of a plurality of the breast images by subject identification.

According to the invention described in Claims 4 and 11, precise discrimination is ensured between the right and left breasts of a subject in the breast radiographed without including a pectoral muscle, wherein the aforementioned discrimination cannot be easily made by subject identification or visual observation.

According to the invention described in Claims 5 and 12, a plurality of control apparatuses are connected to apply processing to the breast image having been captured, and the management apparatus controls the connection between a plurality of the aforementioned control apparatuses and the aforementioned image reading apparatus. This arrangement allows processing to be applied regardless of whether the medical image is a breast image or otherwise.

According to the invention described in Claims 6 and 13, the management apparatus ensures that information is sent to a predetermined control apparatus out of a plurality of control apparatuses. This eliminates the need of cassette registration that has been done in the conventional method, thereby enhancing work efficiency.

According to the invention described in Claims 7 and 14, the management apparatus ensures that information is sent to a control apparatus registering the identification information of a plurality of cassettes to be used for radiographing from a plurality of directions for one patient. This arrangement allows the radiographing technician to use a desired control apparatus, and expands the scope of the freedom in the work of the radiographing technician. This arrangement further correct any error that may occur with respect to radiographing conditions such as the order of registration of cassettes to be used for radiographing, the radiographing direction corresponding to each cassette and the discrimination between the right and left breasts.

According to the invention described in Claims 8 and 15, a plurality of the image reading apparatuses are connected to permit simultaneous reading of a plurality of cassettes, whereby the operation time is minimized.

### BRIEF DESCRIPTION OF THE DRAWINGS

FIG. 1 is a drawing representing the overall structure of the medical radiographing system 100 of the present invention.

FIG. 2 is an external view showing a breast image radiographing apparatus 10.

FIG. 3 is a diagram schematically showing the lateral surface of the radiographing section 1.

FIG. 4 is a perspective view representing a radiographic stand 6 and a cassette C loaded thereon.

FIG. 5 is a diagram schematically showing the lateral surface of the radiographing section 1 when phase contrast radiography is used.

FIG. 6 is a block diagram showing the functional structure of a main body 3.

FIG. 7 is a block diagram showing the functional structure of a control apparatus 30A.

FIG. 8 is a diagram showing an example of loading the data of the radiographing order information storing section 372.

FIG. 9 is a flow diagram representing a process of breast radiographing to be applied in the medical radiographing system 100.

FIG. 10 is a flow diagram representing a process of breast radiographing to be applied in the medical radiographing system 100.

FIG. 11 is a diagram representing an example of a confirmation screen 331 displayed on the display section 33.

FIG. 12 is a flow diagram representing a process of breast radiographing to be applied in the medical radiographing system 100.

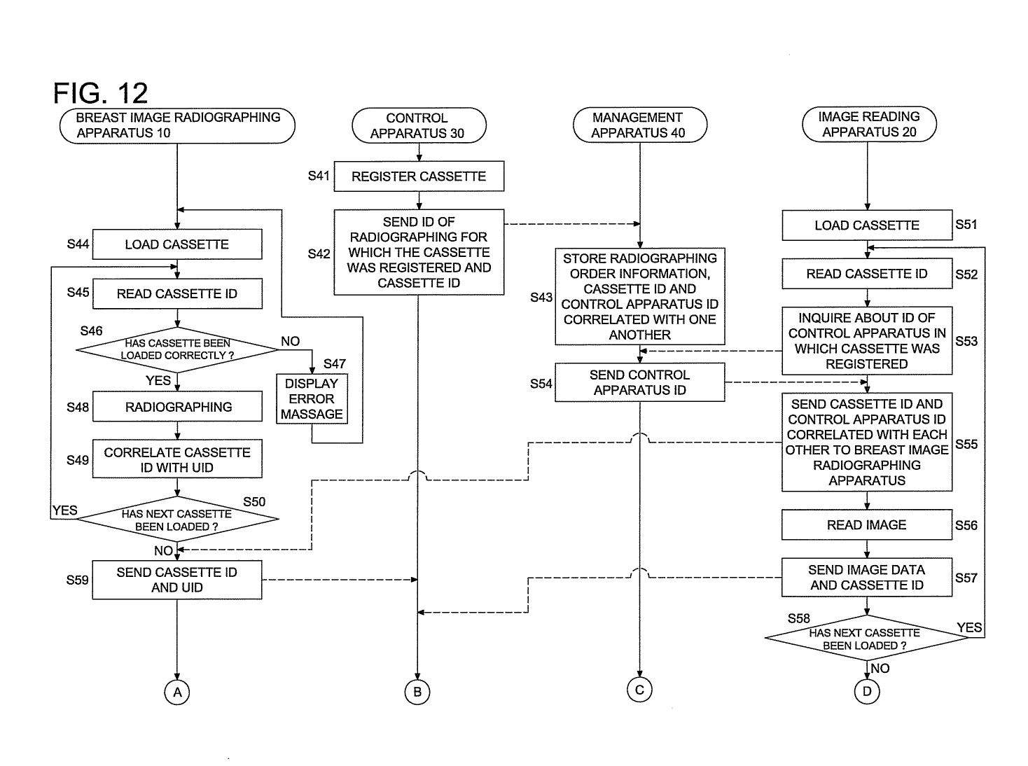

FIG. 13 is a further flow diagram representing a process of breast radiographing to be applied in the medical radiographing system 100.

FIG. 14 is a flow diagram representing a process of changing the correlation of radiographing conditions to be applied in the medical radiographing system 100.

FIG. 15 is a diagram showing an example of the confirmation screen 331 displayed on the display section 33.

FIG. 16 is a diagram representing an example of data storage in the radiographing order information storing section 372.

### DESCRIPTION OF REFERENCE NUMERALS

100 Medical radiographing system
10 breast image radiographing apparatus
20 Image reading apparatus
30 Control apparatus
40 Management apparatus
50 Image output apparatus
1. Radiographing section
2. Column
3. Main body
3a. Control section
3b. Input section
3c. Display section
3d. Interface
3e. Memory
3f. Communication section
4. Support shaft
5. Radiation source
6. Radiographic stand
6a. Cassette holder
6b. Barcode reader
7. Compression plate
20. Image reading apparatus
30. Control apparatus
31. Control section
32. Input section
33. Display section
34. Communication section
35. RAM
36. Image processing section
37. Storing section
371. Unfinished radiographing order list
372. Radiographing order information storing section
373. Original image data storing section
374. Processed image data storing section
38. Barcode reader
C. Cassette
N. Network

### BEST MODE OF CARRYING OUT THE INVENTION

### Embodiment 1

The following describes the structure of the present embodiment:

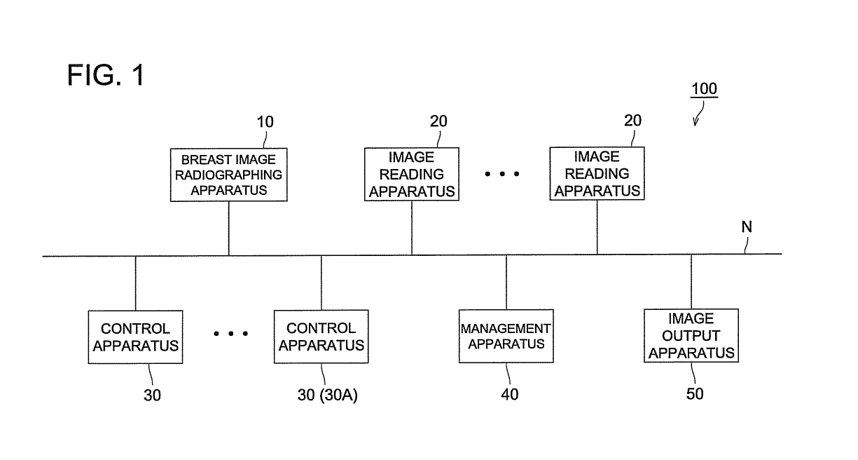

FIG. 1 is a drawing representing the overall structure of the medical radiographing system 100 of the present embodiment. As shown in FIG. 1, the medical radiographing system 100 is provided with a breast image radiographing apparatus 10, a plurality of image reading apparatus 20, a plurality of control apparatuses 30, management apparatus 40 and image output apparatus 50 that can be connected via the network. The medical radiographing system 100 is also provided with a general-purpose radiographing apparatus (not illustrated) for radiographing the region other than the breast. The medical radiographing system 100 is also connected with a hospital information system (hereinafter referred to as “HIS”) and a radiology information system (hereinafter referred to as “RIS”).

The medical radiographing system 100 is capable of both breast radiographing and a general radiographing for radiographing the region other than the breast. Of a plurality of control apparatuses 30, the control apparatus 30A is capable of both the breast radiographing and general radiographing. Other control apparatuses 30 are applicable to only the general radiographing. When the medical radiographing system 100 is used for general radiographing, a plurality of image reading apparatuses 20 and a plurality of control apparatuses 30 are connected for data communication between them. The system configuration wherein connection is made at a ratio of the image reading apparatus 20 to control apparatus 30 being “m” to “n” (each “m” and “n” indicates an integer of two or more) used for general radiographing will be referred to as the general mode. By contrast, when breasts are radiographed by the medical radiographing system 100, breast radiographing section 1, the control apparatuses 30A, and one or more image reading apparatuses 20 designated in advance are connected for data communication. The system configuration wherein connection is made at a ratio of the image reading apparatus 20 to control apparatus 30 being “m” to 1 used at the time of breast radiographing will be referred to as the mammography mode.

Various forms of lines such as LAN (Local Area Network), WAN (Wide Area Network) and the Internet can be used as the network N. The radio communication or infrared ray communication can be used under the permission of a medical institution such as a hospital. However, since the information contains important patient information, the information to be exchanged is preferably encrypted. The DICOM (Digital Image and Communications in Medicine) standard is generally used as the communication system in a hospital. The DICOM MWM (Modality Worklist Management) and DICOM MPPS (Modality Performed Procedure Step) are used for the communication among apparatuses on the aforementioned network N.

The following describes the components of the medical radiographing system 100.

The breast image radiographing apparatus 10 applies radiation to the breast of a patient as the subject, and captures a radiation image of the breast. The present embodiment will be described with reference to an example of using the radiographing apparatus which employs a cassette C. The general radiographing apparatus will also be described with reference to an apparatus which employs a cassette C.

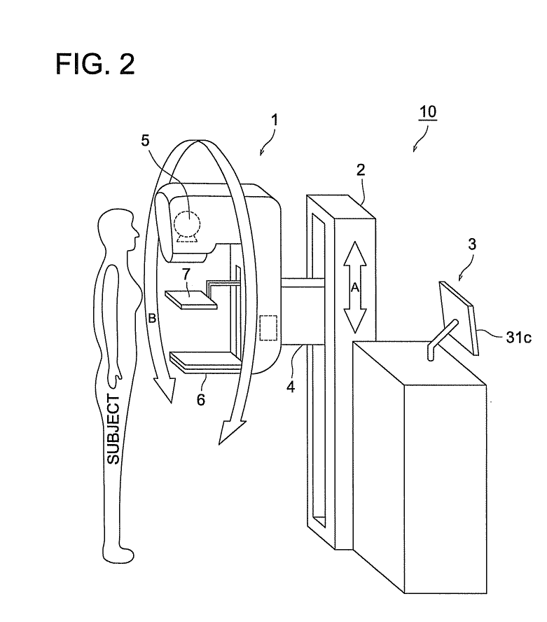

FIG. 2 shows an example of the structure of a breast image radiographing apparatus 10.

As shown in FIG. 2, the breast image radiographing apparatus 10 is provided with a radiographing section 1 for capturing an image by application of radiation, a column 2 and a main body 3. The radiographing section 1 can be moved in the vertical direction (in the direction indicated by an arrow mark A) to ensure that the height is adjusted in conformity to the position of the patient breast. Further, it can be rotated around the support shaft 4 (in the direction indicated by an arrow mark B) in order to change the direction of radiographing. The rotation can be made by manual operation of the radiographing technician, or by operating the main body 3.

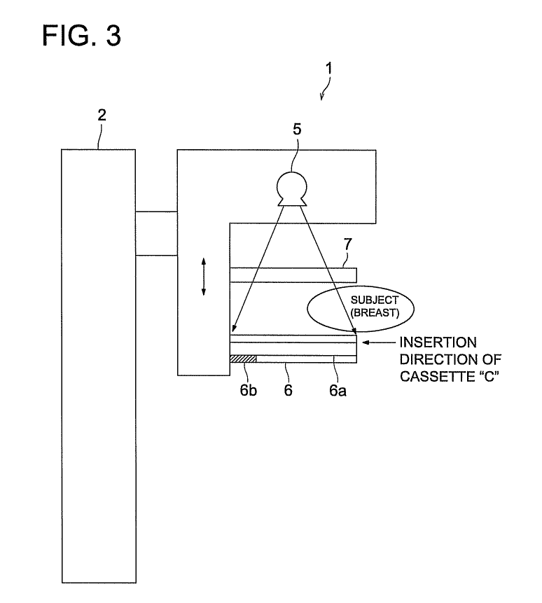

The radiographing section 1 is provided with a radiation source 5 for emitting radiation and a radiographic stand 6 for placing the breasts thereon, wherein the radiation source 5 and radiographic stand 6 are placed face to face with each other. A compression plate 7 is arranged to ensure that the breasts are compressed by sandwiching the breasts placed on the radiographic stand 6. FIG. 3 is side view of the radiographing section 1. As shown in FIG. 3, the radiographing section 1 is so constructed to allow a cassette C to be loaded on the cassette holder 6a of the radiographic stand 6. The cassette C incorporates a stimulable phosphor plate and absorbs the radiation having passed through the subject so that the radiation image is recorded. The cassette C preferably used in the present embodiment is the one designed specifically for breast radiographing wherein one side of the cassette C in contact with the subject is so designed as to capture up to an image of marginal portion of the chest wall of the subject.

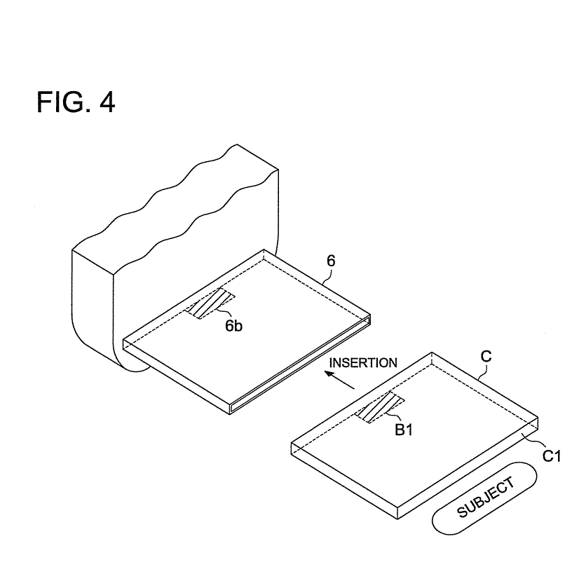

FIG. 4 is a perspective view representing a radiographic stand 6 and a cassette C loaded thereon.

As shown in FIG. 4, the cassette C contains a barcode B1 bearing the identification information (hereinafter referred to as “cassette ID”) for unique identification of the cassette C. In the meantime, the radiographic stand 6 includes a barcode reader 6b for reading the barcode B1 displayed on the cassette C. The barcode B1 of the cassette C is arranged at the center of one side on the rear of the recording surface of the radiation image. The barcode reader 6b of the radiographic stand 6 is mounted at the center of one side on the furthest end in the cassette inserted direction, on the surface wherein the rear side of the cassette C is loaded.

When loading the cassette C on the radiographic stand 6, for example, one side wherein the barcode B1 is attached is inserted face to face with the inlet of the cassette holder 6a by the radiographing technician. This arrangement makes it possible to ensure that the subject recorded on the cassette C can be specified in one direction at all times. Further, one side C1 of the cassette C facing the subject is preferably colored so that the radiographing technician easily identifies the direction wherein the cassette C is inserted. Further, a protrusion or a mark is preferably provided on one side located face to face with the subject (or the other side) so that it can be distinguished from the other long side.

Further, when the barcode B1 attached to the cassette C is read by the barcode reader 6b, it is possible to determine whether or not the cassette C is loaded in a predetermined direction. Thus, in response to the presence or absence of input signal to be inputted from the barcode reader 6b, the breast image radiographing apparatus 10 is allowed to make a quick detection of a possible insertion error of the cassette C.

The barcode B1 and barcode reader 6b can be mounted at any other places than indicated in FIG. 4 provided that, when the cassette C is loaded on the radiographic stand 6, they are aligned with each other, and the radiographing of a subject is not adversely affected.

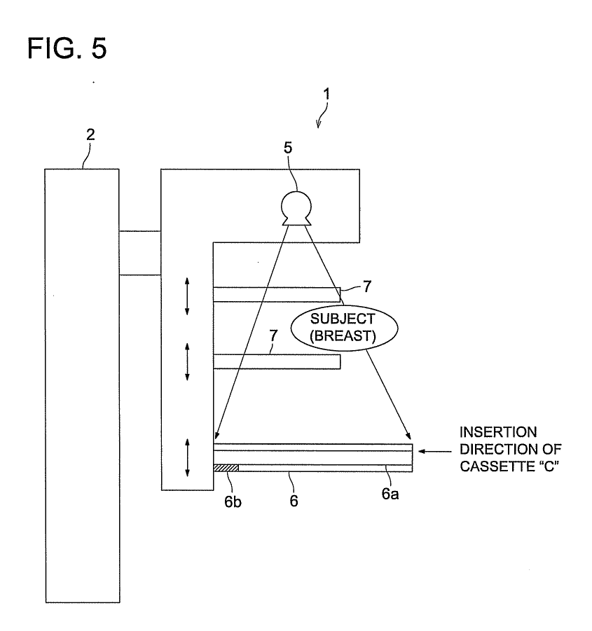

When phase contrast radiographing method is used for radiographing, the radiographing section 1 is structured as shown in FIG. 5. As shown in FIG. 5, the radiographing section 1 includes two compression plates 7, below which the radiographic stand 6 is mounted. Two compression plates 7 and radiographic stand 6 are mounted movably in the vertical direction and their height can be adjusted in conformity to the position of the subject. Further, when the phase contrast radiographing method is used, the size of the cassette C used for radiographing is greater than that of the cassette C used for normal radiographing (size 8″×10″ is used for normal radiographing, and size 14″×17″ is used for the phase contrast radiographing wherein the magnification rate is 175%). Thus, the area of the radiographic stand 6 is increased accordingly.

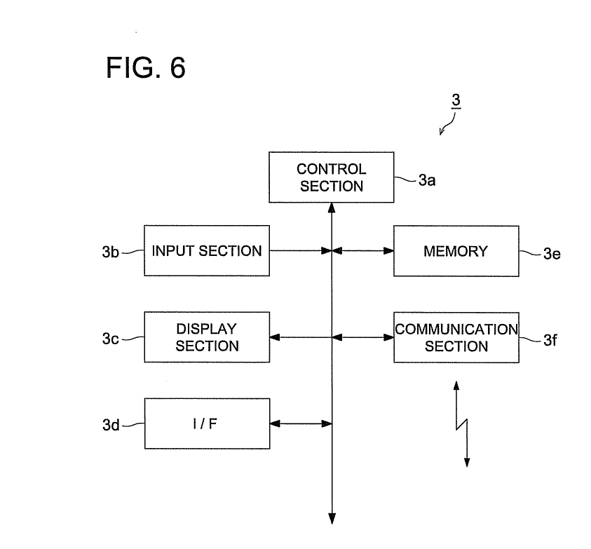

Referring to FIG. 6, the following describes the internal structure of the main body 3.

As shown in FIG. 6, the main body 3 includes a control section 3a, input section 3b, display section 3c, interface 3d, memory 3e and communication section 3f. These components are connected by a bus.

The control section 3a is made of a CPU (Central Processing Unit). Reading various forms of processing program from the memory 3e, the control section 3a collaborates with the program to provide centralized control of the radiographing operations of the components of the breast image radiographing apparatus 10, including adjustment of the timing for application of radiation and radiation exposure dose, for example, in the radiation source 5, and control of the rotation of the radiographing section 1 and the reading of barcodes from the barcode reader 6b. The control section 3a determines if the cassette C is loaded correctly or not, depending on whether or not the barcode B1 has been read from the cassette C loaded on the radiographic stand 6 through the interface 3d, in other words, in response to the presence or absence of the signal input from the barcode reader 6b. Based on the result of decision, if the cassette C has been loaded correctly, the control section 3a controls irradiation from the radiation source 5. Further, the control section 3a acquires information about the time when radiographing is performed (e.g., information about the time of application of the radiation), generates the unique ID (UID) made of information about the time of radiographing, and sends to the control apparatuses 30 the UID correlated to the cassette ID inputted through the interface 3d.

The input section 3b is provided with a key for inputting various forms of setting conditions and outputs the operation signal corresponding to the operated key to the control section 3a. It incorporates various forms of keys as exemplified by the numeric keys for inputting the values of the tube voltage, tube current and the rotary angle of the radiographing section 1.

The input section 3b can be provided with such a key as a radiographing direction key for instructing and inputting the rotary angle of the radiographing section 1. This radiographing direction keys include keys for various radiographing directions such as the vertical direction for the left breast (CC (Cranio-Caudal)-L (left)), the oblique direction for the left breast (MLO (Medio-Lateral Oblique)-L), the vertical direction for the right breast (CC-R (right)) and the oblique direction for the right breast (MLO-R). For example, if an oblique direction key is pressed, the radiographing section 1 is automatically rotated at the rotary angle for radiographing in the internal-external direction.

The display section 3c is provided with an indication display 31c made of an LCD (Liquid Crystal Display), as shown in FIG. 2. Various forms of display information including the input information from the input section 3b and the result of processing by the control section 3a are indicated on the indication display 31c.

The interface 3d is used for connection between the barcode reader 6b and main body 3. The input signal of the barcode B1 read by the barcode reader 6b is inputted into the control section 3a.

The memory 3e is made up of a RAM (Random Access Memory) and ROM (Read Only Memory), and is used to store the breast radiographing programs and various forms of programs including the radiographing program of the present invention. It also stores the result of processing by the control section 3a on the temporary basis, correlates the cassette ID of the cassette C subsequent to radiographing, to the UID, and stores the result on the temporary basis.

The communication section 3f contains the communication interface such as the Network Interface Card (hereinafter referred to as “NIC”) and modem. The communication section 3f sends the cassette ID and UID correlated therewith to the control apparatus 30A in response to the control of the control section 3a.

The image reading apparatus 20 reads the medical image recorded on the cassette C. The image reading apparatus 20 applies the excitation beam to the stimulable phosphor sheet of the cassette C, whereby the photostimulated light emitted from the sheet is converted into electric signals. The image signal having been obtained is then subjected to analog-to-digital conversion, whereby the image data for medical image is obtained. Further, the image reading apparatus 20 is provided with the barcode reader (not illustrated). It reads the cassette ID from the barcode B1 attached to the cassette C, correlates the image data of the medical image to the cassette ID, and sends the result to the control apparatuses 30. In the general mode, the image reading apparatus 20 is connected to a plurality of control apparatuses 30. Thus, the cassette ID corresponding to the image data of the medical image having been read is sent to the management apparatus 40. An inquiry is made to find out any one of the control apparatuses 30 that has registered the cassette ID and that serves as a destination of the image data of the medical image having been read, whereby a destination of the image data of the medical image to be sent is identified. The destination of the image data of the medical image to be sent is identified by obtaining the IP address of the control apparatus 30 (to be described later) from the management apparatus 40. In the mammography mode, the image reading apparatus 20 is connected to the control apparatus 30A, and hence the image data of the medical image having been read is sent to the control apparatus 30A.

When the cassette C is loaded, an error of top and bottom (front and rear) and loading error in the vertical direction is detected by the image reading apparatus 20 by finding that the cassette barcode cannot be read by the barcode reader (not illustrated). Thus, that the direction of loading the cassette C is kept constant (the same orientation) at all times with respect to the image reading apparatus 20.

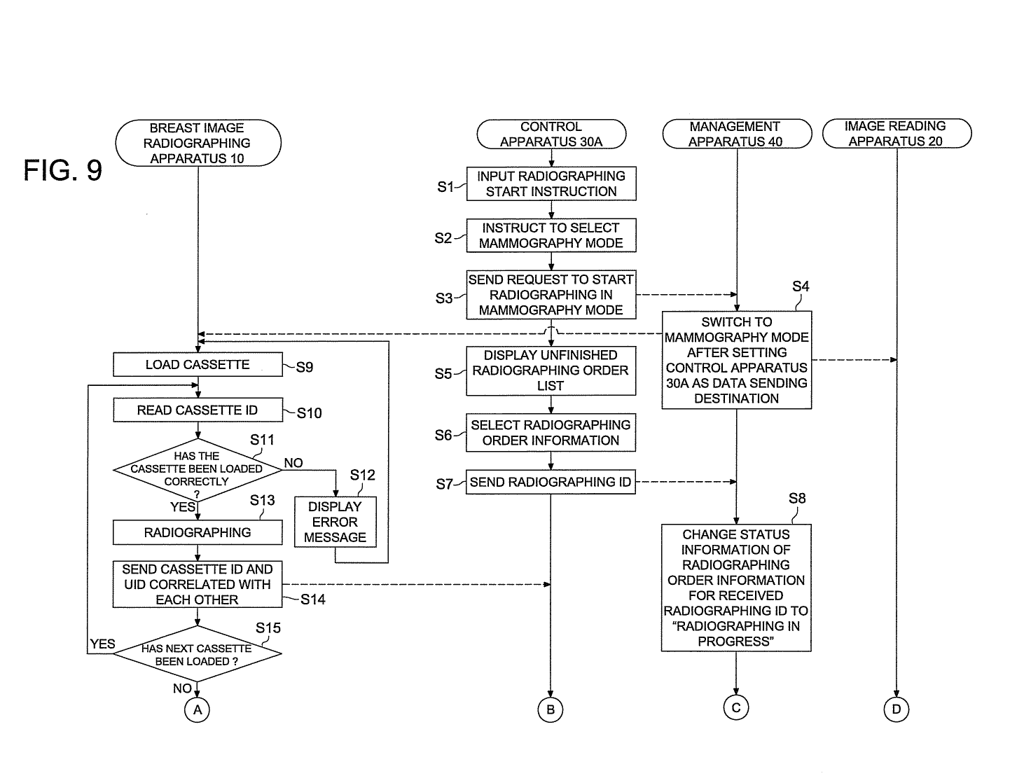

The control apparatus 30 stores the programs (for general radiographing programs) having the functions of determining and registering the correlation between the radiographing order information for general radiographing for radiographing regions other than the breasts, and the cassette ID of the cassette C to be used for radiographing in that order; ensuring that the medical image received from the image reading apparatus 20 is correlated with the radiographing order information, based on the cassette ID; applying processing to the received medical image; and outputting this image. In response to these programs, the control apparatuses 30 applies processing. In addition to the aforementioned programs for general radiographing, the control apparatuses 30A has the programs for mammography mode (mammography program) that provide processing of breast radiographing (processing of breast radiographing on the side of the control apparatus in FIG. 9). The control apparatuses 30 applies processing in response to the program corresponding to the mode selected on the input section 32.

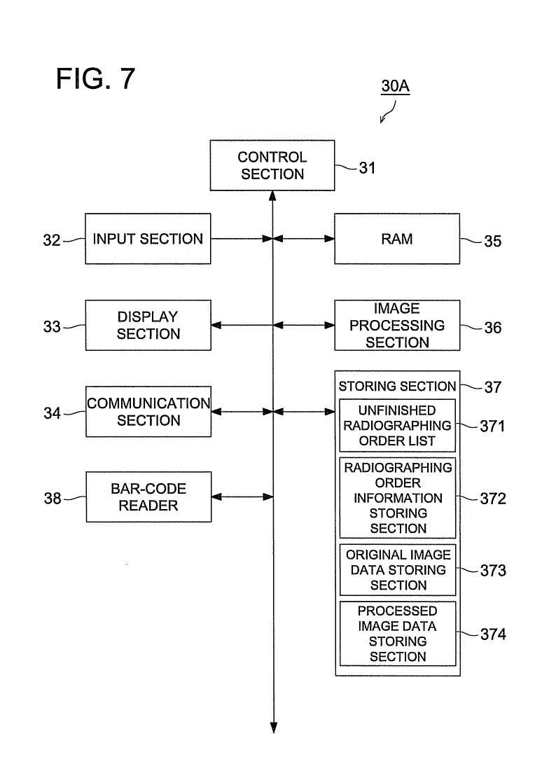

Referring to FIG. 7, the following describes the control apparatus 30A. FIG. 7 is a block diagram showing the structure of the major components of a control apparatus 30A. As shown in FIG. 7, the control apparatus 30A includes a control section 31, input section 32, display section 33, communication section 34, RAM 35, image processing section 36, and storing section 37. They are connected by a bus.

The control section 31 reads the system program stored in the storing section 37, loads it on the work area formed inside the RAM 35, and controls various sections according to the system program. In response to the input instruction from the input section 32, the control section 31 reads out various forms of processing programs including the mammography program, general radiographing program and image processing program stored in the storing section 37, as well as various forms of application programs. They are loaded on the work area so as to execute various forms of processing including the radiographing of the breast image on the control apparatus.

The input section 32 includes a keyboard equipped with a cursor key, numeric input key and various forms of function keys, and a pointing device such as a mouse. The input section 32 outputs to the control section 31 the instruction signal inputted through the key operation on the keyboard and mouse operation. In the input section 32, a touch panel can be arranged on the display screen of the display section 33. In this case, the instruction signal inputted through the touch panel is outputted to the control section 31.

The display section 33 contains such a monitor as an LCD and CRT. In response to the instruction of the display signal inputted in the control section 31, the display section 33 displays the input instruction from the input section 32, and other data.

The communication section 34 is provided with a communication interface such as an NIC or a modem, and controls the communications among the components connected to the network N.

In various forms of processing to be implemented and controlled by the control section 31, the RAM 35 forms a work area for temporarily storing various programs that can be implemented by the control section 31 and that has been read from the storing section 37, as well as the inputted or outputted data and parameters.

In response to the instruction from the control section 31, the image processing section 3 applies various types of image processing to the inputted image data such as the processing of frequency for adjusting the sharpness of the image, the gradation processing for adjusting the contrast, or the processing of dynamic range compression for keeping the image having a wide dynamic range within an easily readable density scope, without reducing the detailed contrast of this image.

The storing section 37 includes an HDD (Hard Disk Drive) and nonvolatile semiconductor memory, and stores the system program to be executed in the control section 31, various forms of processing programs corresponding to this system program including the mammography program, general radiographing program, and image processing program, various forms of application programs and various forms of data. These programs are stored in the form of readable program codes and the control section 31 sequentially executes the operations in response to these program codes.

Further, the storing section 37 contains an unfinished radiographing order list storing section 371, radiographing order information storing section 372, original image data storing section 373, and processed image data storing section 374.

The unfinished radiographing order list storing section 371 stores the unfinished radiographing order information list (hereinafter referred to as “unfinished radiographing order list”) sent from the management apparatus 40.

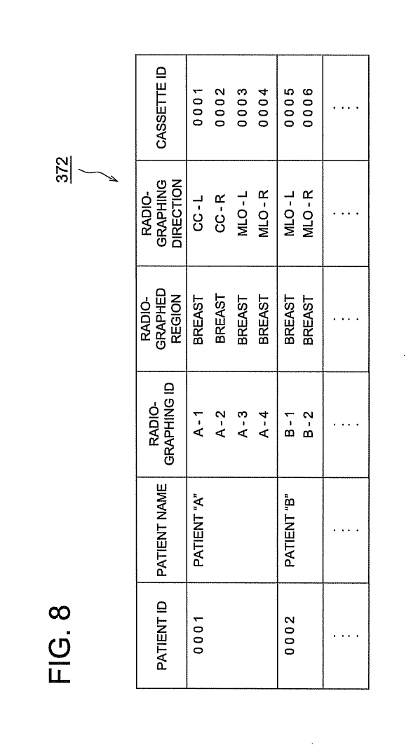

When the cassette ID of the cassette C used for that order has been registered with respect to the radiographing order list in the unfinished radiographing order list, the radiographing order information storing section 372 correlates the radiographing order information with the cassette ID, and stores the result of correlation. FIG. 8 shows an example of storing the data of the radiographing order information storing section 372.

The original image data storing section 373 stores the data of each medical image (original image) obtained from the image reading apparatus 20, and the radiographing ID for identifying the radiographing order information while correlated with each other.

The processed image data storing section 374 stores the data of the processed image having been obtained after processing the medical image obtained from the image reading apparatus 20 and the radiographing ID while correlated with each other.

The barcode reader 38 reads the cassette ID represented by the barcode B1 displayed on the cassette C and outputs it to the control section 31.

When the cassette ID obtained by the reading of the barcode B1 displayed on the cassette C has been correlated with the radiographing order information (hereinafter referred to as “registration of the cassette ID”), the cassette ID together with the identification information of the control apparatus 30A having registered the cassette ID (IP address of the control apparatus 30A in the present embodiment) is sent to the management apparatus 40.

The structure of the control apparatuses 30 other than that of the control apparatus 30A is the same as that of the control apparatus 30A, except that the program for the mammography mode is not stored in the storing section 37.

The management apparatus 40 of FIG. 1 is made up of a computer including a RAM, storing section such as a CPU, ROM, HDD storing the programs, input section, display section and communication section. It is a server to control the entire medical radiographing system 100. The management apparatus 40 acquires the radiographing order information registered on the RIS or HIS at predetermined time intervals, correlates this information with the “unfinished” status information, stores and controls it in the radiographing order DB (Data Base) of the storing section, sends the unfinished radiographing order information to each of the control apparatuses 30 through the network at predetermined time intervals, and updates the unfinished radiographing order list stored in the control apparatuses 30. The status information is sent to the management apparatus 40 from the control apparatuses 30 in conformity to the progress of the work in response to each of the radiographing order information items. This status information and the control apparatus ID of the currently working control apparatuses 30 are correlated with the radiographing order information and are stored in the radiographing order database, whereby the progress of the work in response to each radiographing order information is managed. In this case, description will be made on the assumption that the progress is managed by the management apparatus 40 according to three statuses “Unfinished Radiographing”, “Radiographing in Progress” and “Finished Radiographing”.

The management apparatus 40 has a function of switching the system configuration (system mode) of the medical radiographing system 100 between the general mode and mammography mode. When selecting the general mode, the IP addresses of all the control apparatuses 30 are set on the image reading apparatus 20 as the IP addresses of the data that can be sent. When selecting the mammography mode, the IP address of the specific control apparatus 30A as the IP addresses of the data destination is set on the breast image radiographing apparatus 10 and predetermined image reading apparatuses 20 (one up to “m” apparatuses can be predetermined, but two apparatuses are normally sufficient with consideration given to the relationship between the radiographing cycle and image reading cycle).

The image output apparatus 50 outputs the processed image having been distributed by the image server 4. A monitor for display outputting, or a film output apparatus for film outputting can be used as the image output apparatus 50.

The following describes the operation in the present embodiment:

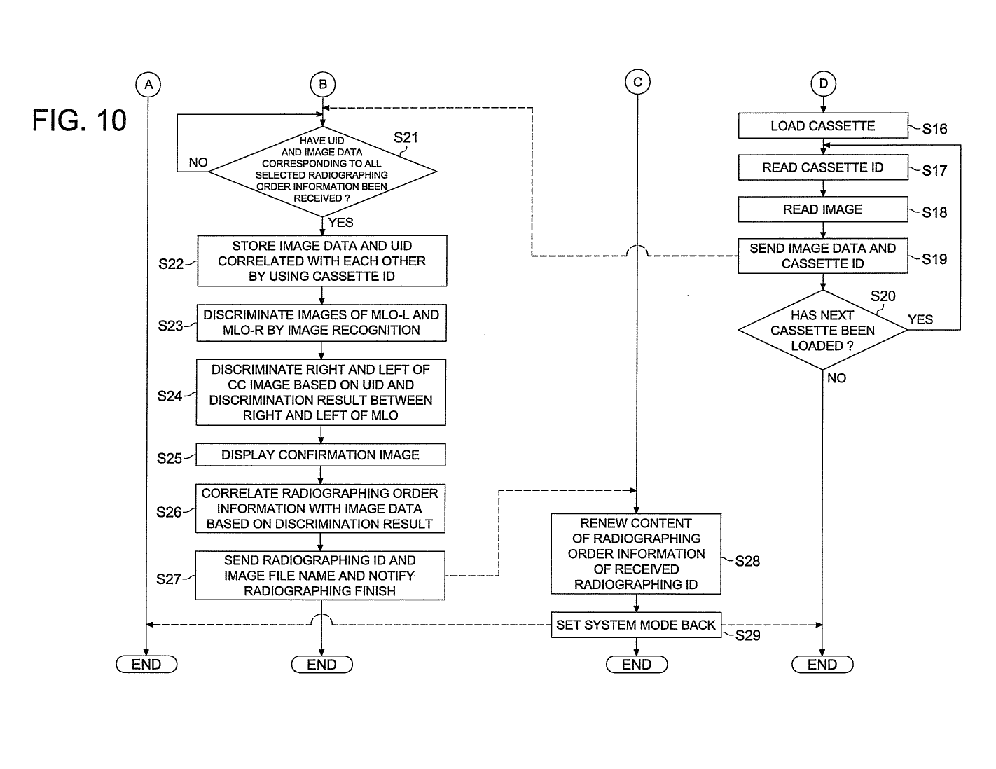

FIGS. 9 and 10 are flow diagrams showing the first embodiment of breast radiographing processing performed by the medical radiographing system 100. As shown in the process of breast radiographing, radiographing request information is inputted in the control apparatus 30A, and the instruction is given to start processing in the mammography mode. Then the system mode of the medical radiographing system 100 is switched over to the mammography mode and the designated image reading apparatus 20 can communicate with only the specific control apparatus 30A out of control apparatuses 30. Accordingly, the process of image reading can be performed, without the need of registering the correlation between the cassette C to be used and radiographing order information before radiographing (no need of registering the cassette).

In the processing of breast radiographing shown in FIGS. 9 and 10, one patient is subjected to four radiographing operations under four radiographing conditions (regions and directions for radiographing) such as radiographing of the left breast in the oblique direction (MLO (Medio-Lateral oblique)-L), radiographing of the right breast in the oblique direction (MLO-R), radiographing of the left breast in the vertical direction (CC (Cranio-Caudal)-L), and radiographing of the right breast in the vertical direction (CC-R).

In FIG. 9, the radiographing technician stands in front of the control apparatus 30A and operates the input section 32 to input the radiographing start instruction. When the input section 32 has been operated by the radiographing technician to input the radiographing start instruction in the control apparatus 30A (Step S1), the display section 33 shows a mode selection screen to prompt selection of the system mode between the general and the mammography modes. When the mammography mode has been selected on this mode selection screen, and an instruction has been given to start radiographing in the mammography mode (Step S2), a request for radiographing start in the mammography mode is sent to the management apparatus 40 (Step S3). When the management apparatus 40 has received the request for radiographing start in the mammography mode from the control apparatus 30A, the control apparatus 30A is set as the data destination on the breast image radiographing apparatus 10 and predetermined image reading apparatus 20, and the system mode is switched to the mammography mode (Step S4).

In the control apparatus 30A, the unfinished radiographing order list data is read from the unfinished radiographing order list storing section 371, and is displayed on the display section 33 (Step S5). The radiographing technician selects the radiographing order information of the patient as a subject to be radiographed, from this unfinished radiographing order list on display through the input section 32. In this case, radiographing order information under four radiographing conditions is selected for one patient as a subject to be radiographed. When the radiographing order information has been selected through the input section 32 (Step S6), the radiographing ID of the radiographing order information having been selected is sent to the management apparatus 40 (Step S7).

When the management apparatus 40 has received the radiographing ID from the control apparatus 30A, the status of the radiographing order information of the radiographing ID having been received is switched over to “Radiographing in Progress” (Step S8).

Upon completion of selecting the radiographing order information of the subject to be radiographed in the control apparatus 30A of Steps S1 through S7, the radiographing technician carries a plurality of cassettes C to be used for radiographing and goes into the radiographing room wherein the breast image radiographing apparatus 10 is installed. He then loads one of the cassettes C on the radiographic stand 6 of the breast image radiographing apparatus 10. In the breast image radiographing apparatus 10, when the cassette C has been loaded on the radiographic stand 6 (Step S9), the barcode B1 is read by the barcode reader 6b, and the cassette ID is captured into the apparatus (Step S10). If the cassette C is not loaded in the correct direction, in other words, if it is not loaded in such a way that the barcode reader 6b and barcode B1 are located face to face with each other without the barcode B1 being read (Step S11: NO), an error message representing a loading error is shown on the display section 3c (Step S12). Then the process goes back to the Step S9. If the cassette C is correctly loaded (Step S11: YES), radiographing is performed according to the input instruction on the input section 3b by the radiographing technician (Step S13). Then the UID based on time axis representing the time of radiographing is generated at the time of radiographing. The UID is correlated with the cassette ID read in Step S10 and is sent to the control apparatus 30A having been set as the destination (Step S14). When the next cassette has been loaded (Step S15: YES), the process goes back to the Step S10, and processing in Steps S10 through S14 is repeated.

Upon completion of radiographing in Steps S9 through S15, the radiographing technician loads the cassette C having been used, on a desired one of the image reading apparatuses 20 having been set to the mammography mode. In the breast image radiographing apparatus 10, one patient is subjected to radiographing under four different radiographing conditions. The cassette C having been used can be loaded on the image reading apparatus 20 at the end of every radiographing operation; alternatively, the cassettes C having been radiographed can be loaded collectively upon completion of a series of radiographing operations. At this time, there is no need of worrying about the order of loading the cassettes C. If there are a plurality of image reading apparatuses 20 set to the mammography mode, a series of reading operation of the radiographed images for one patient can be performed using either one of these apparatuses or a plurality of them.

In FIG. 10, when the cassette C has been loaded on the image reading apparatus 20 (Step S16), the cassette ID is read by the barcode reader (Step S17). Further, the image recorded on the loaded cassette C is read (Step S18), and the obtained data and the cassette ID read in Step S17 are correlated with each other so that they are sent to the control apparatus 30A predetermined as the destination (Step S19). When the next cassette C has been loaded (Step S20: YES), the process goes back to the Step S17, and processing in Steps S17 through S19 is repeated.

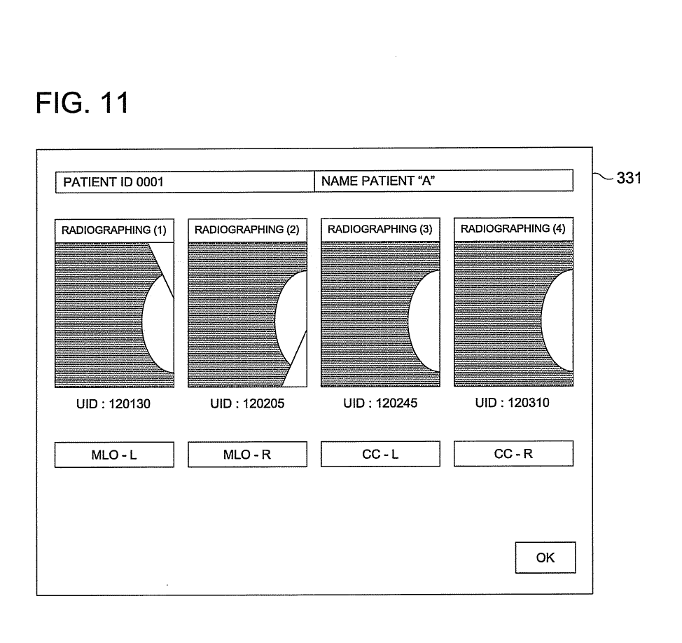

When reading of the image data shown in Steps S16 through S20 has been completed, and the control apparatus 30A has been received the UID from the breast image radiographing apparatus 10 and the image data from the image reading apparatus 20 for all the radiographing orders under four radiographing conditions with respect to the patient as the subject to be radiographed (Step S21: YES), the received UID and image data are correlated with each other using the cassette ID as a key. The UID is recorded on the header of the image data and is stored in the original image data storing section 373 (Step S22). Further, each of the four pieces of image data is identified on the screen and the images bearing the radiographing conditions of MLO-L and MLO-R are selected (Step S23). The image with the radiographing direction of MLO corresponds to the breast image captured while including the pectoral muscle (images of RADIOGRAPHING (1) and RADIOGRAPHING (2) in FIG. 11). The shape of the skin line differs according to whether the breast as the subject to be radiographing is right or left. As described above, the relative position between the cassette C and the subject is determined at the time of radiographing. Accordingly, for the MLO image, the right and left of the radiographed breasts can be identified by visual observation.

In the above description, the right and left of the radiographed breasts are identified by visual observation. Without the present invention being restricted thereto, the breast image can be analyzed by the control apparatuses 30 and automatic discrimination can be applied. For the breast image to be captured, all the cassettes C are loaded on the breast image radiographing apparatus 10 in one and the same direction and are loaded on the image reading apparatus 20 in one and the same direction. Accordingly, the breast images captured in one and the same direction at one and the same region to be radiographed should be similar to each other. Thus, the pattern images for the MLO right and left breasts are prepared in advance, and discrimination can be made by comparison between these pattern images and the breast images having been captured.

To put it more specifically, comparison is made between the pattern image of the MLO left breast and the captured breast image. If there is agreement between the pattern image of the MLO left breast and the captured breast image, the breast image is determined as “L”. If there is no agreement between the pattern image of the MLO left breast and the captured breast image, the process goes to the step of comparison with the pattern image of the MLO right breast. If there is agreement between the pattern image of the MLO right breast and the captured breast image, the breast image is determined as “R”. As described above, the right and left distinction of the breast image having been radiographed is automatically made. This eliminates the need of the MLO breast image being specified by the radiographing technician, whereby the burden of the radiographing technician is reduced.

When automatic discrimination is performed in the processing of Step S23, it is not always necessary to employ one and the same radiographing condition (e.g., a predetermined side of the cassette C is located on the chest wall) to ensure that the chest wall is located on a predetermined side of the rectangular image having been read. This allows the positional relationship between the cassette C and chest wall to be determined as desired. To be more specific, any of the four sides of the cassette C can be placed face to face with the chest wall. It should be noted, however, that this may increase the capacity of the pattern image to be stored in advance to perform automatic discrimination or may increase the time of identifying the pattern.

When no restriction is imposed on the direction of the cassette C to be loaded at the time of radiographing, right and left reversal (mirroring) processing of the breast image is required to find agreement between the right and left breast images in the MLO breast image. The direction of loading the CR cassette on the image reading apparatus 20 is kept constant by the aforementioned barcode reading function (an alarm of barcode reading error is issued except for the predetermined direction of loading). Accordingly, agreement between the right and left of the breast images cannot be achieved by rotation processing of the breast image alone. This is utilized to determine the right and left of the breast image.

To be more specific, make a comparison between the pattern image of the left breast and the captured breast image. If there is agreement between the two as a result of comparison, the breast image is determined as “L”. If not, the pattern image of the left breast is turned 90 degrees successively, or the pattern images turned 90 degrees each is prepared in advance. This pattern image is again compared with the captured breast image. This procedure is repeated four times. If no agreement is found at all, the process goes to the step of comparing the pattern image of the right breast. The same procedure as that in the case of the aforementioned pattern image of the left breast is used to makes a comparison between the pattern image of the right breast and the captured breast image, whereby the right and left of the breast images are determined.

Going back to FIG. 10, the order of radiographing is determined according to the UID assigned to each image, and the right and left of the breasts of the subject in other images (CC images in this case) is determined according to the order of radiographing, and the right and left MLO images having been discriminated (Step S24). The display section 33 shows the confirmation screen 331 that displays the images arranged according to the order of radiographing and the result of determining the direction of radiographing, and discriminating right and left breasts of each image (Step S25). In this case, the control section 31 of the control apparatus 30A serves as a radiographing order determination device, and right and left discrimination device.

Normally, the breasts are pressed by the compression plate at the time of radiographing the breasts, and this gives pain to the patient. Accordingly, continuous radiographing of the same breast is not performed. To be more specific, when radiographing four breast images, radiographing is done alternately on the right and left (i.e., left→right→left→right or right→left→right→left). Thus, the right and left of the subject breasts in the CC image can be determined based on the UID assigned to each piece of image data and the right and left discrimination of the MLO image.

In the aforementioned example, it is also possible to arrange such a configuration that, after automatic discrimination of the right and left of at least one MLO image, the right and left of the remaining breast images are discriminated based on the result of this discrimination and the order of radiographing.

FIG. 11 shows an example of a confirmation screen 331 displayed in Step S25. The confirmation screen 331 shows four breast images according to the order of radiographing based on the UID. Further, the right and left discrimination and the directions of radiographing correlated with each image (MLO-L, MLO-R, CC-L and CC-R) are displayed. This confirmation screen 331 allows the user to examine the result of automatic discrimination on the direction of radiographing and right and left breasts.

When the confirmation has been completed and the OK button of the input section 3b has been pressed, the radiographing order information selected as the object for radiographing in Step S6 is correlated with the image data wherein there is agreement between the direction of radiographing in the radiographing order information, and the direction of radiographing having been discriminated and the result of right and left discrimination, according to the result of discriminating the direction of radiographing and right and left breasts (Step S26). For example, the radiographing order information is correlated with the image file name and is stored. The radiographing ID subsequent to radiographing, and image file name are sent to the management apparatus 40. Then the completion of radiographing is notified (Step S27).

In the management apparatus 40, the status information of the radiographing order information of the radiographing ID having been received is updated. The radiographing order information is correlated with the image file name of the image data, and is stored (Step S28). Further, the system mode is switched over to the general mode. To be more specific, the system is switched over to the “m:n” system configuration wherein all the image reading apparatuses 20 can communicate with all the control apparatuses 30 (Step S29), whereby this processing terminates.

Upon termination of the aforementioned process of breast radiographing, each image is subjected to the processing of gradation, frequency enhancement or the like in the image processing section 36 of the control apparatuses 30. Based on the result of discriminating the direction of radiographing and right and left breasts, the composite image of the MLO right and left breast images and that of the CC right and left breast images are created. The composite image data is sent to the image output apparatus 50 and is outputted from the image output apparatus 50.

In breast radiographing in periodic physical examination or the like, two images of MLO-L and MLO-R are often captured for one patient. If a seemingly abnormal shadow has been detected, four images of MLO-L, MLO-R, CC-L and CC-R are captured. In the case of images of MLO-L and MLO-R alone, discrimination between the MLO-L and MLO-R can be made by image recognition or visual observation, as described above.

In the medical radiographing system 100, as described above, when the radiographing technician inputs the start of radiographing from a specific control apparatus 30A out of a plurality of control apparatuses 30, the control apparatus 30A displays the mode selection screen on the display section 33. When the mammography mode is selected from this mode selection screen, switching over to the mammography mode is specified. This causes the system configuration to be switched over to a configuration wherein the breast image radiographing apparatus 10 and image reading apparatus 20 having been specified in advance can communicate only with the control apparatus 30A, in other words, wherein the relationship between the image reading apparatus 20 and control apparatuses 30 is m to 1. When the radiographing technician has selected the radiographing order information under four radiographing conditions of MLO-L, MLO-R, CC-L and CC-R for one patient from the control apparatus 30A, and has loaded the cassette C on the radiographic stand 6 of the breast image radiographing apparatus 10, the breast image radiographing apparatus 10 reads the cassette ID of the cassette C having been loaded, and generates the UID for indicating the time of radiographing using this cassette C. The cassette ID and UID are correlated with each other and are sent to the control apparatus 30A. After radiographing, the radiographing technician loads the cassette C having been used, on the specified apparatus out of a plurality of image reading apparatus 20. Then the image reading apparatus 20 reads the cassette ID and sends the cassette ID together with the read image data to the control apparatus 30A. The control apparatus 30A correlates the image data and the UID having received, using the cassette ID as a key, and discriminates between the MLO-L image and MLO-R image by image recognition. Based on the result of this discrimination and the order of radiographing represented by the UID, the control apparatus 30A discriminates between the CC-L and CC-R images, and displays the result on the confirmation screen 331.

Thus, the order of radiographing and image recognition makes it possible to achieve discrimination between the right and left breasts of the subject and discrimination of the direction of radiographing in a plurality of breast images for one patient. This arrangement eliminates the possibility of wrong discrimination of the right and left breasts of the subject in the captured breast image, or wrong discrimination of the direction of radiographing which may be caused by correlation error by the radiographing technician between the radiographing conditions and cassette C to be used. When breast images are to be captured under a plurality of radiographing conditions for one patient, the radiographing technician is relieved of a burden of having to remember the radiographing conditions for each of the cassettes C, unlike in the conventional method. He is also relieved of the burden of loading the cassettes C having been used on the image reading apparatus 20 in the order of radiographing for the purpose of being read. Thus, this arrangement contributes to a substantial reduction in the workload of the radiographing technician.

At the time of breast radiographing, the system configuration is switched over to the m:1 system configuration on the temporary basis. This eliminates the need of previously registering the correlation between the cassette C and radiographing order information, and enhances work efficiency.

The description of the above embodiment represents one of examples wherein the medical radiographing system 100 of the present invention is preferably applied, without the present invention being restricted thereto.

For example, the aforementioned embodiment has been described with reference to the example wherein the barcode B1 attached to the cassette C is read by the barcode reader 6b, whereby discrimination is made to determine whether or not the cassette C is correctly loaded on the cassette holder 6a of the radiographic stand 6. However, the present invention is not restricted thereto. For example, it is also possible to arrange such a configuration that the cassette holder 6a and cassette C are provided with a fitting portion and fitted portion that can be fitted with each other, so that the direction of loading the cassette C on the radiographic stand 6 is restricted to one direction. This arrangement allows the breast image to be recorded on the cassette C loaded in the correct direction, and ensures correct identification of the direction of the breast image in the subsequent processing steps.

In the aforementioned embodiment, the MLO-L image and the MLO-R image are identified by image recognition. It is also possible to arrange such a configuration that, in the control apparatuses 30, four breast images are displayed in parallel on the screen according to the order of radiographing in response to the UID, and the MLO-L image and/or MLO-R image are identified by the visual observation of the user. The result of discrimination is inputted in the system.

### Embodiment 2

The following describes the structure of the second embodiment:

FIG. 1 is a conceptual diagram representing the overall structure of the breast radiographing system 100 in the present embodiment. As show in FIG. 1, the breast radiographing system 100 is connected with the breast image radiographing apparatus 10, one or more image reading apparatuses 20, one or more control apparatuses 30, management apparatus 40, image output apparatus 50, and others in such a way that mutual communication is possible via the network N. In this case, the breast radiographing system 100 is further connected with a Hospital Information System (hereinafter referred to as “HIS”) for managing the information in a hospital or a Radiology Information System (hereinafter referred to as “RIS”) for managing the information in the department of radiology.

The following describes the components of the breast radiographing system 100.

The breast image radiographing apparatus 10 applies radiation to the breasts of a patient as the subject, whereby the breast is radiographed. The present embodiment will be described with reference to the example wherein a radiographing apparatus using a cassette C is employed.

The breast image radiographing apparatus 10, radiographic stand 6 and radiographing section 1 are the same as those described with reference to the first embodiment, and will not be described here.

The following describes the internal structure of the main body 3 with reference to FIG. 6:

As shown in FIG. 6, the main body 3 is made up of a control section 3a, input section 3b, display section 3c, interface 3d, memory 3e and communication section 3f. These components are connected via the bus.

The control section 3a, input section 3b, display section 3c, interface 3d and memory 3e are the same as those described with reference to the first embodiment, and will not be described here.

The communication section 3f includes a communication interface such as the Network Interface Card (hereinafter referred to as “NIC”) and modem. The communication section 3f sends the cassette ID and the UID correlated therewith to the control apparatuses 30, in response to the control of the control section 3a.

The image reading apparatus 20 reads the medical image (breast image in the present embodiment) recorded on the cassette C. The image reading apparatus 20 applies excitation light to the stimulable phosphor sheet of the cassette C. Thus, the photostimulated light emitted from this sheet is converted into electric signal, and the image signal obtained therefrom is subjected to analog-to-digital conversion, whereby medical image data is obtained. Further, the image reading apparatus 20 has a barcode reader (not illustrated), which reads the cassette ID from the barcode B1 attached to the cassette C. Then the medical image data and cassette ID are correlated with each other, and are sent to the control apparatuses 30. When the cassette is loaded on the image reading apparatus 20, a possible loading error including wrong top/bottom (front/rear) positions or wrong position in the vertical direction is detected by the failure to read the cassette barcode by the barcode reader (not illustrated). This arrangement ensures that the loading direction of the cassette C is kept always constant (the same) with reference to the image reading apparatus 20.

The control apparatus 30 receives the list of the unfinished radiographing order information (including the instruction information on the radiographing conditions such the patient information on the patient to be radiographed, the region to be radiographed, and direction of radiographing) from the management apparatus 40, and registers the correlation between the radiographing order information and the cassette ID of the cassette C used for radiographing of this order. Further, the medical image received from the image reading apparatus 20 (breast image in this case) is correlated with the radiographing order information based on the cassette ID, and the received medical image is processed, whereby breast images are composed in a predetermined format.

The following describes the control apparatus 30 with reference to FIG. 7. FIG. 7 is a block diagram showing the functional structure of the control apparatus 30. As shown in FIG. 7, the control apparatus 30 includes a control section 31, input section 32, display section 33, communication section 34, RAM 35, image processing section 36, storing section 37 and barcode reader 38. These components are connected by a bus.

The control section 31 reads the system program stored in the storing section 37 and loads it in the network area formed in the RAM 35, whereby various sections are controlled according to the system program. The control section 31 reads various forms of programs including the breast radiographing processing program and image processing program, and various forms of application programs stored in the storing section 37. These programs are loaded on the work area to execute processing of breast radiographing (to be described later) processing on the control apparatus.

The input section 32, display section 33, communication section 34, RAM 35 and image processing section 36 are the same as those described with reference to the first embodiment, and will not be described here.

The storing section 37 is made up of the HDD (Hard Disk Drive) and nonvolatile semiconductor memory. It stores the system program executed by the control section 31, various forms of processing programs including the breast radiographing program corresponding to the system program, and the image processing program, various forms of application programs, and various forms of data. These various forms of programs are stored in the form of a readable program code, and the control section 31 sequentially executes operations according to the program code.

The storing section 37 contains an unfinished radiographing order list storing section 371, radiographing order information storing section 372, original image data storing section 373 and processed image data storing section 374.

The unfinished radiographing order list storing section 371 stores the unfinished radiographing order list sent from the management apparatus 40 (hereinafter referred to as “unfinished radiographing order list”).

When the cassette ID of the cassette C to be used for the order is registered with respect to the radiographing order information in the unfinished radiographing order list, the radiographing order information and cassette ID are correlated with each other, and are stored in the radiographing order information storing section 372. FIG. 8 shows an example of storing the data of the radiographing order information storing section 372.

The data of each medical image (original image) obtained from the image reading apparatus 20, and the radiographing ID for identifying the radiographing order information are correlated with each other, and are stored in the original image data storing section 373.

The processed image data obtained by applying image processing to each of the medical images obtained from the image reading apparatus 20 and radiographing ID are correlated with each other and are stored in the processed image data storing section 374.

The barcode reader 38 reads the cassette ID shown by the barcode B1 displayed on the cassette C, and the result is sent to the control section 31.

The management apparatus 40 is made up of a computer including a CPU, storing section such as a ROM, RAM, HDD storing the programs, input section, display section and communication section. It is a server to control the entire breast radiographing system 100. The management apparatus 40 acquires the radiographing order information registered on the RIS or HIS at predetermined time intervals, correlates this information with the “unfinished” status information, stores and controls it in the radiographing order DB (Data Base) of the storing section, sends the unfinished radiographing order information to each of the control apparatuses 30 through the network at predetermined time intervals, and updates the unfinished radiographing order list stored in the control apparatuses 30. The status information is sent to the management apparatus 40 from the control apparatuses 30 in conformity to the progress of the work in response to each of radiographing order information items. This status information and the control apparatus ID of the currently working control apparatuses 30 are correlated with the radiographing order information and are stored in the radiographing order database, whereby the progress of the work in response to each of the radiographing order information items is managed. In this case, description will be made on the assumption that the progress is managed by the management apparatus 40 according to three statuses “Unfinished Radiographing”, “Radiographing in Progress” and “Finished Radiographing”.

The image output apparatus 50 outputs the processed image having been distributed by the image server 4. A monitor for display outputting, or a film output apparatus for film outputting can be used as the image output apparatus 50.

The following describes other operation in the present embodiment:

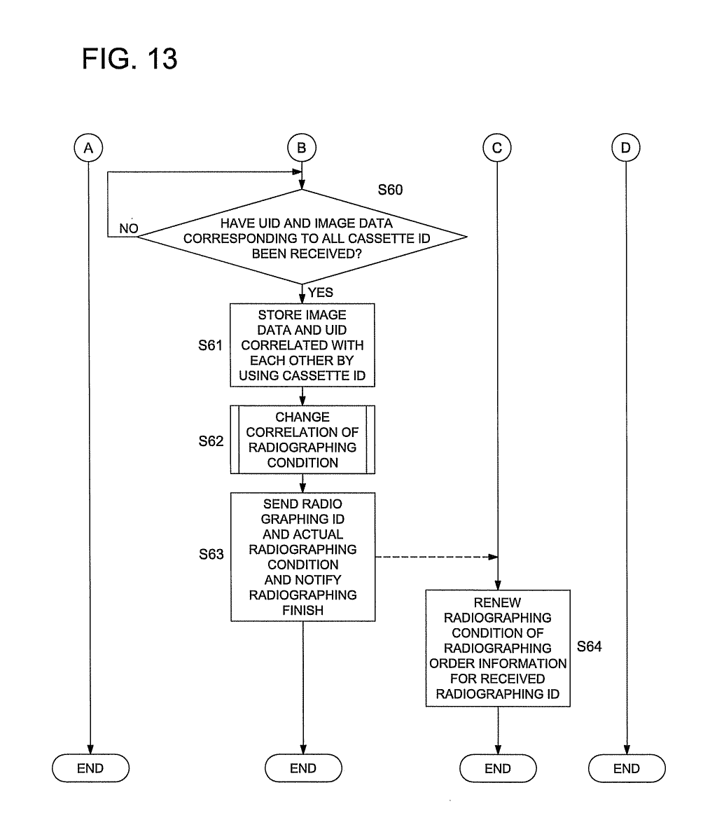

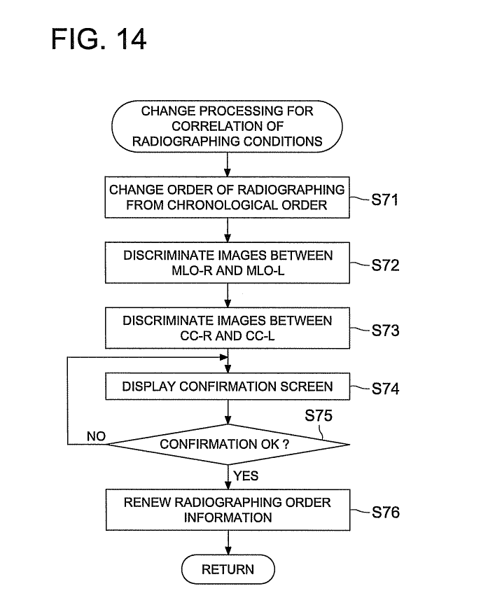

FIGS. 13 and 14 are flow diagrams showing the second embodiment of breast radiographing performed by the breast radiographing system 100. In the processing of breast radiographing in FIGS. 13 and 14, one patient is subjected to four radiographing operations under four radiographing conditions (regions and directions for radiographing) such as radiographing of the left breast in the oblique direction (MLO (Medio-Lateral Oblique)-L), radiographing of the right breast in the oblique direction (MLO-R), radiographing of the left breast in the vertical direction (CC (Cranio-Caudal)-L), and radiographing of the right breast in the vertical direction (CC-R). The difference of the second embodiment of FIGS. 13 and 14 from the first embodiment is that radiographing of the subject is started after the cassette ID previously assigned to the cassette C and the radiographing order information have been correlated with each other.

In the first place, the radiographing technician carries a plurality of cassettes C to be used for a series of breast radiographing operations for one subject, and goes in front of a desired control apparatus 30 to register the cassette. In the control apparatus 30, in response to the operation of the input section 32 and barcode reader 38 by the radiographing technician, the radiographing order information for the subject to be radiographed is selected and the cassette is registered (Step S41). To put it more specifically, when the radiographing technician operates the input section 32 to enter the instruction to display the unfinished radiographing order list, in the control apparatus 30 the data of the unfinished order list is read from the unfinished radiographing order list storing section 371 and is displayed on the display section 33. Then the radiographing technician selects the radiographing order information of the subject to be radiographed from the displayed unfinished order list through the input section 32, and the barcode reader 38 is operated to read the barcode B1 displayed on the cassette C to be used, according to the selected radiographing order information. Then the cassette ID having been read is correlated with the selected radiographing order information by the control apparatus 30. The result is stored in the radiographing order information storing section 372, whereby the cassette is registered. In the registration of the cassette, the cassette ID of the cassette C used in radiographing is registered for the radiographing order information of each of the four radiographing conditions for one subject to be radiographed.

Upon completion of cassette registration, the radiographing ID of the radiographing order information wherein the cassette has been registered, and the cassette ID are correlated with each other by the control apparatus 30, and the result is sent to the management apparatus 40 (Step S42). Upon receipt of the radiographing ID and cassette ID from the control apparatus 30, the cassette ID and the control apparatus ID of the source from which information has been sent are correlated with the radiographing order information of the radiographing ID having been received in the radiographing order database to be stored, by the management apparatus 40 (Step S43). Then the status information of the radiographing order information is changed to “Radiographing in Progress”.

Upon completion of cassette registration in the control apparatus 30, the radiographing technician carries a plurality of cassettes C having been registered, and goes to the radiographing room wherein the breast image radiographing apparatus 10 is installed. In principle, the cassette C is loaded on the radiographic stand 6 of the breast image radiographing apparatus 10 according to the registered information. In the breast image radiographing apparatus 10, when the cassette C has been loaded on the radiographic stand 6 (Step S44), the barcode B1 is read by the barcode reader 6b, and the cassette ID is acquired (Step S45). If the cassette C is not correctly loaded, in other words, if it is not loaded in such a way that the barcode reader 6b is located face to face with the barcode B1, and the cassette ID cannot be read (Step S46: NO), an error message appears on the display section 3c to notify loading error (Step S47). Then the process goes back to the Step S44. If the cassette has been correctly loaded (Step S46: YES), radiographing is implemented in response to the instruction inputted into the input section 3b by the radiographing technician (Step S48). At the time of radiographing, the UID based on time axis to represent the time of radiographing is generated, and is correlated with the cassette ID read in Step S45, and is stored in the memory 3e (Step S49). When the net cassette has been loaded (Step S50: YES), the process goes back to the Step S45, whereby the procedures of Steps S45 through S50 are repeated.

Upon completion of radiographing, the radiographing technician loads the cassette C having been used, on a desired one of the image reading apparatuses 20. In the breast image radiographing apparatus 10, one patient is subjected to radiographing under four different radiographing conditions. The cassette C having been radiographed can be loaded on the image reading apparatus 20 at the end of every radiographing operation; alternatively, the cassettes C having been radiographed can be loaded collectively upon completion of a series of radiographing operations. At this time, there is no need of worrying about the order of loading the cassettes C. A series of reading operation of the radiographed images for one patient can be performed using either one of these apparatuses or a plurality of them.

When the cassette C has been loaded on the image reading apparatus 20 (Step S51), the cassette ID is read by the barcode reader (Step S52). Then the cassette ID is sent to the management apparatus 40 and an inquiry is made to identify the ID of the control apparatus that has registered the cassette (IP address) (Step S53).

When the inquiry about the control apparatus ID from the image reading apparatus 20 has been received by the management apparatus 40, the radiographing order database is searched to get the control apparatus ID of the control apparatus 30 having registered the cassette ID which has been received, and the control apparatus ID is sent to the image reading apparatus 20 in return (Step S54). When the image reading apparatus 20 has received the control apparatus ID from the management apparatus 40, the cassette ID and control apparatus ID are correlated with each other and the result is sent to the breast image radiographing apparatus 10 (Step S55). This is followed by the step of reading the image recorded on the loaded cassette C (Step S56). Then the obtained image data and the cassette ID read in Step S52 are correlated with each other, and the result is sent to the control apparatus 30 of the control apparatus ID sent back from the management apparatus 40 (Step S57). When the next cassette has been loaded (Step S58: YES), the process goes back to the Step S52, and the procedures of Steps S52 through S57 are repeated.

In the meantime, when the breast image radiographing apparatus 10 has received the cassette ID and control apparatus ID from the image reading apparatus 20, the UID stored in the memory 3e while correlated with the received cassette ID is read, and this read UID and the cassette ID not having been received are correlated with each other, and are sent to the control apparatus of the control apparatus ID which has not received from the image reading apparatus 20 (Step S59).

When the control apparatuses 30 has received the UID from the breast image radiographing apparatus 10 and the image data from the image reading apparatus 20 for all the radiographing orders under four radiographing conditions for the subject to be radiographed (Step S60: YES), the received UID, image data and radiographing order information are correlated with one another using the cassette ID as a key, and the UID is recorded on the header of the image data. This is recorded on the original image data storing section 373 (Step S61).

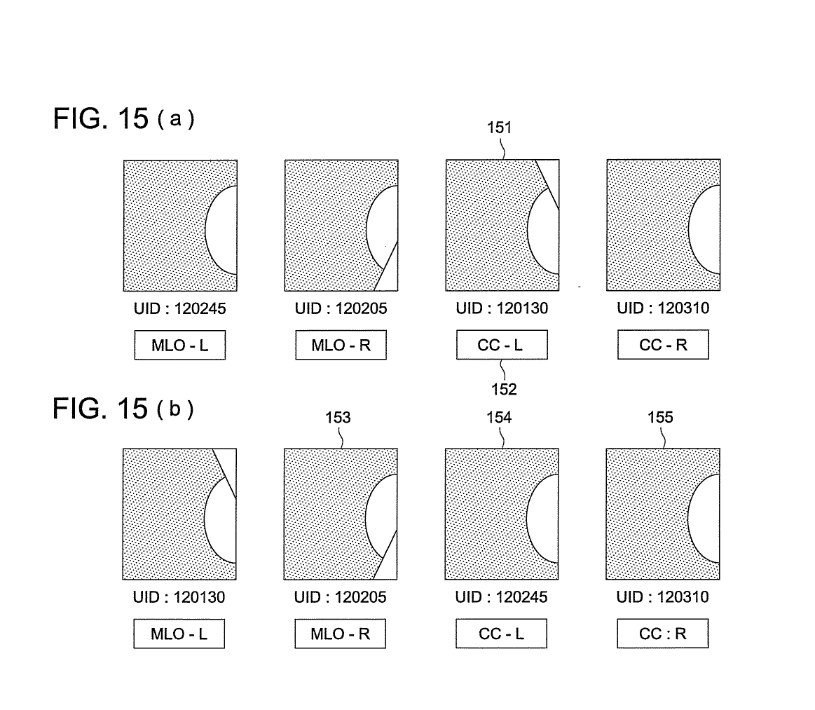

In Step S62, correlation between the radiographing order information and image data is changed as required. Processing in Step S62 is used when the cassette that is correlated with the radiographing order information is apparently different from the cassette used for radiographing, for example, for the image having been captured, when the radiographing condition CC 152 is correlated with the image 151 that should have been captured while correlated with the “MLO” radiographing order information (FIG. 15 (a)). Processing in Step S62 is also used when the radiographing technician feels anxious about the possibility of having used a wrong cassette.

Referring to FIG. 14, the following describes the details of the procedure of changing the correlation in Step S62. It is also possible to arrange such a configuration that the procedure of changing the correlation is performed automatically after the procedure of Step S61 in FIG. 13, or performed by the radiographing technician operating the input section 32 of the control apparatus 30A.

In Step S71, these four breast images are rearranged in the order of radiographing, based on the UID correlated with each of four breast images which are the radiographed images of the subject, stored in the original image data storing section 373. To be more specific, the UIDs correlated with the cassette ID are issued in the order of radiographing, as radiographing time information. Thus, when the images are rearranged according to the UIDs, they are arranged in the chronological order or in the reversed order thereof. In this case, the control section 31 of the control apparatus 30A works as a radiographing order decision device.

In Step S72, the breast images rearranged in Step S71 are each subjected to image recognition, similarly to the case of the process of image discrimination in Step S23 of FIG. 11, whereby images of MLO-L and MLO-R as radiographing condition are discriminated.

All the breast images to be captured are loaded on the breast image radiographing apparatus 10 in one and the same direction, and are loaded on the image reading apparatus 20 in one and the same direction. Accordingly, the breast images in one and the same direction and region to be radiographed should be similar to each other. Thus, the pattern images of the right and left breasts of MLO are prepared in advance and comparison between these pattern images and the breast images having been captured, whereby discrimination is made between the two.

To put it more specifically, comparison is made between the pattern image of the left breast and the radiographed breast image. If there is agreement between the pattern image of the left breast and the radiographed breast image after comparison, the breast image is determined as “L”. If there is no agreement between the pattern image of the left breast and the radiographed breast image, the process goes to the step of comparison with the pattern image of the right breast. If there is agreement between the pattern image of the right breast and the radiographed breast image, the breast image is determined as “R”

When automatic discrimination is performed, use of the same radiographing condition so that the chest wall is located at a predetermined side in the rectangular image having been read (the predetermined side of the cassette C is located at the check wall) is not always essential, in other words, the positional relationship between the cassette C and check wall is free. To be more specific, any of the four sides of the cassette C can be set face to face with the check wall. However, for the purpose of automatic discrimination, there may be an increase in the volume of the pattern image to be stored in advance or an increase in the time required for pattern recognition.

When there is no restriction to the orientation of the cassette C to be loaded at the time of radiographing, mirroring of the breast image is required for the purpose of ensuring agreement between the right and left breast images in the MLO breast image. The direction of loading of the CR cassette on the image reading apparatus 20 is kept constant by the barcode reading function (an alarm of barcode reading error will be issued except in a predetermined loading direction). Accordingly, right and left agreement cannot be achieved by mirroring of the breast image alone. This is utilized for discrimination between the right and left breast images.

To be more specific, in the first place, comparison is made between the pattern image of the left breast and the radiographed breast image. If there is agreement between the pattern image of the left breast and the radiographed breast image after comparison, the breast image is determined as “L”. If there is no agreement, the pattern image of the left breast is turned 90 degrees successively. Alternatively, the pattern images having been turned 90 degrees successively are prepared in advance. This pattern image is compared again with the radiographed breast image. This procedure is repeated four times. If there is no agreement at all, the process goes to the step of comparing the pattern image of the right breast. Similarly to the case of the aforementioned pattern image of the left breast, comparison is made between the pattern image of the right breast and the radiographed breast image, whereby discrimination is made between the right and left of the breast images.

In Step S73, the right and left distinction of the two remaining CC breast images is made based on the right and left distinction determined in Step S72. Normally, the breasts are pressed by the compression plate at the time of radiographing the breasts, and this gives pain to the patient. Accordingly, continuous radiographing of the other side of the breasts is not performed. To be more specific, when radiographing four breast images, radiographing is done alternately on the right and left (i.e., left→right→left→right or right→left→right→left). Thus, the right and left of the subject breasts in the CC image can be discriminated based on the UID assigned to each piece of image data and the right and left discrimination of the MLO image. For example, as shown in FIG. 15 (b), the CC breast image 154 to be radiographed immediately after the MLO right breast image 153 is the left breast image, namely, CC-L, and the CC breast image 155 to be radiographed immediately after the CC-L is CC-R.

In the processing of Steps S72 and S73, the control section 31 of the control apparatus 30A serves as a right and left discrimination device.

In Steps S74, the display section 33 displays the confirmation screen 331 showing the image arranged in the order of radiographing, and the direction of radiographing and the right and left distinction result for each image. The confirmation screen 331 is the same as the example given in FIG. 11. The right and left distinction and direction of radiographing (MLO-L, MLO-R, CC-L and CC-R) are displayed corresponding to each image.

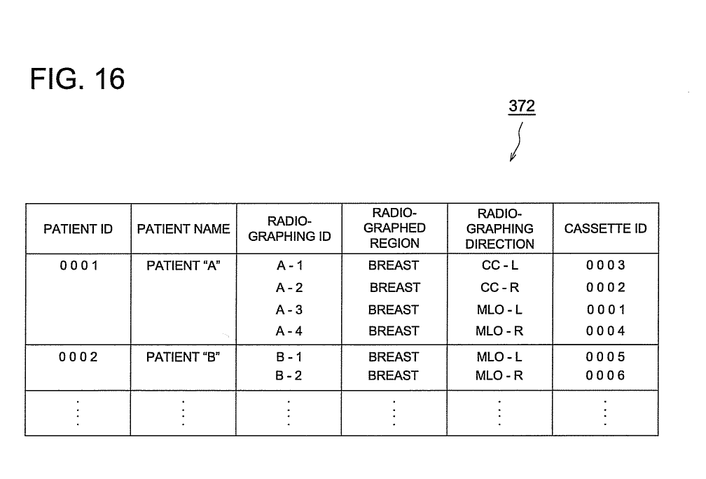

When the OK button of the input section 3b has been pressed upon completion of confirmation in Step S74 (Step S75: YES), the radiographing order information storing section 372 is searched for each data using the UID as a key. The radiographing condition of the radiographing order information corresponding to each piece of image data is rewritten as shown in FIG. 16, in response to the direction of radiographing and the result of right and left distinction in Steps S72 and S74 (Step S76).

Going back to FIG. 14, the radiographing ID after radiographing, the actual radiographing direction for the corresponding cassette and the right and left distinction of the breast of the subject are sent to the management apparatus 40, whereby completion of radiographing is notified (Step S63).

The management apparatus 40 updates the status information of the radiographing order information of the radiographing ID and the radiographing conditions (direction of radiographing and right and left distinction of the breast of the subject) having received. This processing then is completed (Step S64).

Upon completion of the aforementioned breast image radiographing processing, in the image processing section 36 of the control apparatus 30, image processing such as processing of gradation and frequency enhancement is applied to each image. The composite image of the MLO right and left breast images and the composite image of the CC right and left breast images are generated, based on the result of discriminating the direction of radiographing and right and left breasts. The composite image data is sent to the image output apparatus 50, from which it is outputted.

In breast radiographing in periodic physical examination or the like, two images of MLO-L and MLO-R are often radiographed for one subject. If a seemingly abnormal shadow has been detected, four images of MLO-L, MLO-R, CC-L and CC-R are captured. In the case of images of MLO-L and MLO-R alone, discrimination between the MLO-L and MLO-R can be made by image recognition or visual observation, as described above.

In the medical radiographing system 100, as described above, when the radiographing technician has selected the radiographing order information under four imaging conditions of MLO-L, MLO-R, CC-L and CC-R for a subject from a desired control apparatus out of a plurality of control apparatuses 30, and has registered the cassette ID of the cassette used in the four radiographing operations, the breast image radiographing apparatus 10 reads the cassette ID of the loaded cassette and generates the UID showing the time of radiographing using the cassette C. The cassette ID and UID are correlated with each other and are sent to the control apparatuses 30 having registered the cassette. After radiographing, when the radiographing technician has loaded the cassette C subsequent to radiographing, on a desired one of a plurality of image reading apparatuses 20, the image reading apparatus 20 reads the cassette ID and sends it together with the image data having been read, to the control apparatus 30 having registered. The control apparatus 30 correlates the received image data and the UID with the radiographing order information, using the cassette ID as a key, and the image is displayed. The displayed information is confirmed. If the radiographing technician has detected any confusion of cassettes, he specifies the automatic image discrimination function. The right and left distinction of the subject breasts in a plurality of breast images for one subject and distinction of radiographing direction can be achieved by the radiographing order and image recognition. Accordingly, when the right and left distinction of the subject breasts and the direction of radiographing in the breast image captured have been incorrectly recognized due to the correlation error between the radiographing condition and the cassette C used, caused by the radiographing technician, incorrect recognition can be automatically corrected.

The radiographing technician is allowed to register a cassette from a desired one among a plurality of control apparatuses 30. This arrangement allows the radiographing technician to work using the nearest control apparatus 30, with the result that work efficiency is enhanced. Further, the radiographing technician is also allowed to read the cassette using a plurality of image reading apparatuses 20, hence the work efficiency is further improved.

Further, the detailed structures and operations of the components of the breast radiographing system 100 can be modified as desired, without departing from the technological spirit and scope of the invention claimed.

1. A method of discriminating between right and left breast images in a plurality of breast images obtained by radiographing each of right and left breasts of a subject, the method comprising the steps of:
acquiring information about time of radiographing for each of the plurality of breast images stored, the breast images having been correlated with the information about time of radiographing in advance;
determining an order of radiographing for each of the plurality of breast images, based on the acquired information about time of radiographing;
discriminating between the right and left breasts of the subject in a pectoral muscle image captured while including a pectoral muscle, among the plurality of the breast images; and
discriminating between the right and left breasts in each of a plurality of other breast images, based on the order of radiographing, and the distinction of the right and left breasts in the pectoral muscle image having been determined.

2. The method of discriminating between the right and left breast images described in claim 1,
wherein, radiographing for right and left breasts of one subject in an order of alternately radiographing the right and left breasts from a plurality of different radiographing directions, including a radiographing direction of an oblique direction for an image including a pectoral muscle is conducted by using:
a breast radiographing system including:
a radiographing apparatus for radiographing to capture a breast image by applying radiation and recording the breast image in a cassette;
a control apparatus for processing the captured breast image; and
an image reading apparatus for reading the breast image recorded in the cassette and sending the read breast image to the control apparatus;
wherein the radiographing apparatus, the control apparatus and the image reading apparatus are connected so as to permit data transmission and reception;
wherein, in the radiographing apparatus, identification information of the cassette to be used for each radiographing is read, the read cassette identification information is correlated with the information about time of radiographing, and the correlated information is sent to the control apparatus;
wherein, in the image reading apparatus, when the cassette used for radiographing has been loaded, the identification information of the loaded cassette as well as the breast image recorded on the cassette is read and the read cassette identification information and the breast image are sent correlated with each other to the control apparatus; and
wherein, in the control apparatus, upon receipt of a plurality of captured breast images of the subject and a plurality of pieces of the information about time, each of the plurality of received breast images is correlated with each of the plurality of pieces of the information about time based on the cassette identification information, an order of radiographing for the plurality of breast images is determined based on the plurality of pieces of the received information about time of the breast images, discrimination is made between the right and left breasts of the subject in the pectoral muscle image captured while including a pectoral muscle, among the plurality of the breast images, and discrimination is made between the right and left of the breasts of the subject in the breast image captured from other direction based on the order of radiographing for the plurality of breast images and the right and left distinction of the breast in the pectoral muscle image having been determined.

3. The method of discriminating between the right and left breast images described in claim 1,
wherein, by identifying the subject of the plurality of breast images, the right and left breasts of the subject are discriminated in at least one breast image captured while including a pectoral muscle, among the plurality of breast images.

4. The method of discriminating between the right and left breast images described in claim 1,
wherein the discrimination between right and left breasts is made on an assumption that a side of a breast among right and left breasts which is in a breast image which has the radiographing order following or followed by a radiographing order of the breast image including a pectoral muscle is different from a side of a breast among right and left breasts which is in the breast image including a pectoral muscle.

5. The method of discriminating between the right and left breast images described in claim 2,
wherein a plurality of control apparatuses for processing the breast images captured by the radiographing apparatus are connected with the image reading apparatus, and a management apparatus controls a connection between the plurality of control apparatuses and the image reading apparatus.

6. The method of discriminating between the right and left breast images described in claim 5,
wherein the management apparatus ensures that the breast image is sent to a predetermined control apparatus among the plurality of control apparatuses.

7. The method of discriminating between the right and left breast images described in claim 5,
wherein the management apparatus ensures that the breast image is sent to the control apparatus wherein the identification information of a plurality of the cassettes to be used for radiographing of the subject from the plurality of radiographing directions has been registered.

8. The method of discriminating between the right and left breast images described in claim 5,
wherein a plurality of image reading apparatuses are connected to the breast radiographing system.

9. A breast radiographing system comprising:
a radiographing apparatus for radiographing to capture a breast image by applying radiation and recording the breast image on a cassette;
a control apparatus for processing the captured breast image; and
an image reading apparatus for reading the breast image recorded on the cassette and sending the read breast image to the control apparatus;
wherein the radiographing apparatus, the control apparatus and the image reading apparatus are connected so as to permit data transmission and reception,
wherein, radiographing is conducted for each of right and left breasts of one subject in an order of alternately radiographing the right and left breasts from a plurality of different radiographing directions, including a radiographing direction of an oblique direction for an image including a pectoral muscle,
wherein, in the radiographing apparatus, identification information of the cassette to be used for each radiographing is read, the read cassette identification information is correlated with information about time of radiographing, and the correlated information is sent to the control apparatus, and
wherein, in the image reading apparatus, when the cassette used for radiographing has been loaded, the identification information of the loaded cassette as well as the breast image recorded on the cassette is read and the read cassette identification information and the breast image are sent correlated with each other to the control apparatus,
the control apparatus including:
a receiving device for receiving a plurality of captured breast images of the subject and a plurality of pieces of information about time;
a radiographing order determining device for correlating each of the plurality of received breast images with each of the plurality of pieces of information about time based on the cassette identification information, and determining an order of radiographing for the plurality of the breast images having been received; and
a right and left discrimination device for discriminating between the right and left breasts of the subject in a pectoral muscle image captured while including a pectoral muscle, among the plurality of the breast images, and for discriminating between the right and left of the breasts of the subject in the breast images captured from other directions based on the order of radiographing for a plurality of breast images and the right and left distinction of the breast having been determined in the pectoral muscle image captured while including a pectoral muscle.

10. The breast radiographing system described in claim 9,
wherein the right and left discrimination device includes a subject identification device for identifying the subject of the plurality of breast images, and discriminates between the right and left breasts of the subject in the breast images captured while including a pectoral muscle, among the plurality of breast images.

11. The breast radiographing system described in claim 9,
wherein the right and left discrimination device performs right and left discrimination on an assumption that a side of a breast among right and left breasts of the subject which is in the breast image which has been captured from other direction and which has the radiographing order following or followed by the radiographing order of the breast image including a pectoral muscle is different from a side of a breast among right and left breasts which is in the breast image including a pectoral muscle.

12. The breast radiographing system described in claim 9, further comprising a management apparatus,
wherein a plurality of control apparatuses for processing a breast image having been captured by the radiographing apparatus are connected with the image reading apparatus, and the management apparatus controls a connection between the plurality of control apparatuses and the image reading apparatus.

13. The breast radiographing system described in claim 12,
wherein the management apparatus ensures that the breast image is sent to a predetermined control apparatus among the plurality of control apparatuses.

14. The breast radiographing system described in claim 12,
wherein the management apparatus ensures that the breast image is sent to the control apparatus wherein the identification information for a plurality of cassettes to be used for radiographing of the subject from the plurality of radiographing directions has been registered.

15. The breast radiographing system described in claim 12,
wherein a plurality of image reading apparatuses are connected to the breast radiographing system.
